(19) United States
(12) Patent Application Publication
(10) Pub. No.: US 2014/0142462 A1
(43) Pub. Date: May 22, 2014

(54) PERIPHERAL TEMPERATURE MEASURING

(71) Applicants: Alberto Fazzi, Eindhoven (NL); Martijn Schellekens, Eindhoven (NL)

(72) Inventors: Alberto Fazzi, Eindhoven (NL); Martijn Schellekens, Eindhoven (NL)

(73) Assignee: KONINKLIJKE PHILIPS N.V., EINDHOVEN (NL)

(21) Appl. No.: 14/123,247

(22) PCT Filed: Jun. 14, 2012
(86) PCT No.: PCT/IB2012/053002

§ 371 Date: Dec. 2, 2013

Related U.S. Application Data
(60) Provisional application No. 61/497,301, filed on Jun. 15, 2011.

(51) Int. Cl. A61B 5/01; A61B 5/00

(52) U.S. Cl. CPC A61B 5/01; A61B 5/704
USPC 600/549

(57) ABSTRACT

Systems and methods for contact-less, non-invasive determination of peripheral temperature of a subject use a thermal exchanger to locally modulate the temperature of a structure in engagement with the subject, such as a subject support structure that supports the subject or an item worn by the subject. A thermal sensor can be used to monitor a thermal response of the subject and to determine the peripheral temperature. Additionally or alternatively, the core body temperature may be determined.

This application is related to U.S. patent application Ser. No. 12/531,313, entitled “Methods and Devices for Measuring Core Body Temperature,” and filed Mar. 15, 2007, as well as U.S. Pat. No. 3,933,045, entitled “Temperature Measurement,” and filed Apr. 30, 1971. All of the related patents and applications are hereby incorporated by reference into the present application in their entirety.

The present disclosure pertains to a method and apparatus for measuring a temperature, and, in particular, measuring peripheral temperature in neonates.

It is well known to measure a temperature of a subject. Specifically, the core body temperature and the peripheral temperature are important measures for diagnostic purposes, including the evaluation of circulatory problems, perfusion, thermoregulation issues, heat/cold stress and infections.

Accordingly, it is an object of one or more embodiments of the present invention to provide a measuring system for contact-less, non-invasive determination of a temperature of a subject. The system comprises a body of engagement which may be configured to support a subject thereon; one or more sensors that generate one or more output signals conveying measurements related to a temperature of the subject, wherein one or more sensors are carried by the body of engagement; a thermal exchanger configured to exchange thermal energy with the body of engagement and/or the subject; and one or more processors configured to execute computer program modules, the computer program modules comprising a control module and a parameter determination module. The control module is configured to control the thermal exchanger to modulate a structural temperature of the body of engagement at or near a point of engagement between the subject and the body of engagement. The parameter determination module is configured to determine a peripheral temperature of the subject based on a thermal response of the subject to the modulation of the structural temperature based on the one or more output signals.

It is yet another aspect of one or more embodiments of the present invention to provide a method of contact-less, non-invasive determination of a temperature of a subject. The method comprises engaging a subject with a body of engagement; generating one or more output signals conveying measurements related to a temperature of the subject; modulating a structural temperature of the body of engagement at or near a point of engagement between the subject and the body of engagement; and determining a peripheral temperature of the subject based on a thermal response of the subject to the modulation of the structural temperature.

It is yet another aspect of one or more embodiments to provide a system configured for contact-less, non-invasive determination of a temperature of a subject. The system comprises means for engaging a subject with a body of engagement; means for generating one or more output signals conveying measurements related to a temperature of the subject; means for modulating a structural temperature of the body of engagement at or near a point of engagement with the subject; and means for determining a peripheral temperature of the subject based on a thermal response of the subject to the modulation of the structural temperature.

These and other objects, features, and characteristics of the present embodiments, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of any limits.

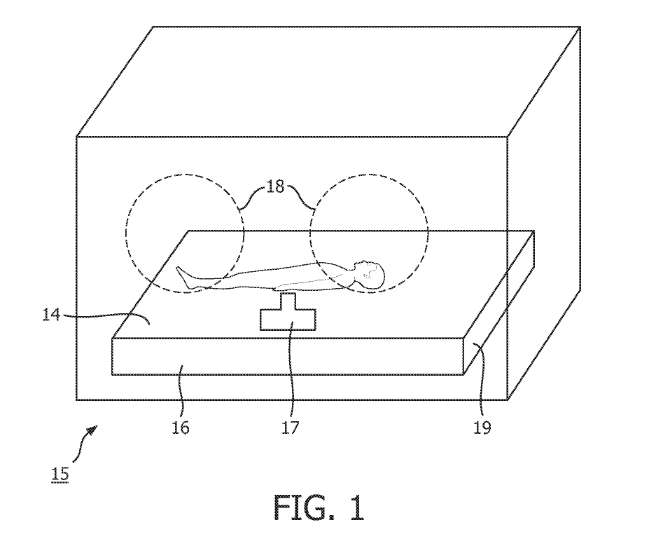

FIG. 1 schematically illustrates a temperature sensor integrated within an incubator;

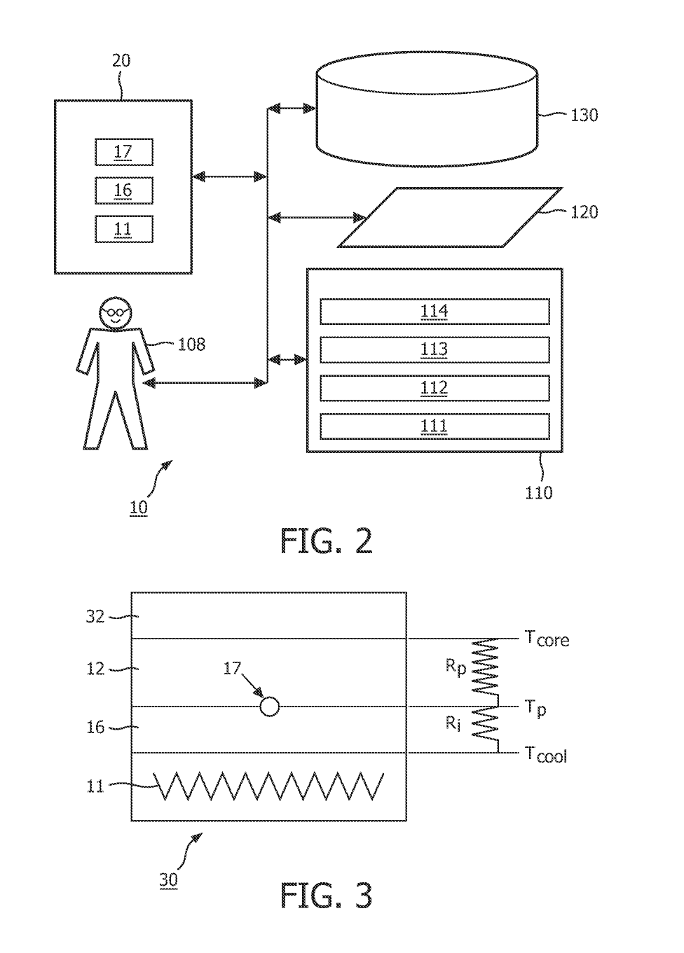

FIG. 2 schematically illustrates a measuring system in accordance with one or more embodiments;

FIG. 3 schematically illustrates a thermal model in accordance with one or more embodiments;

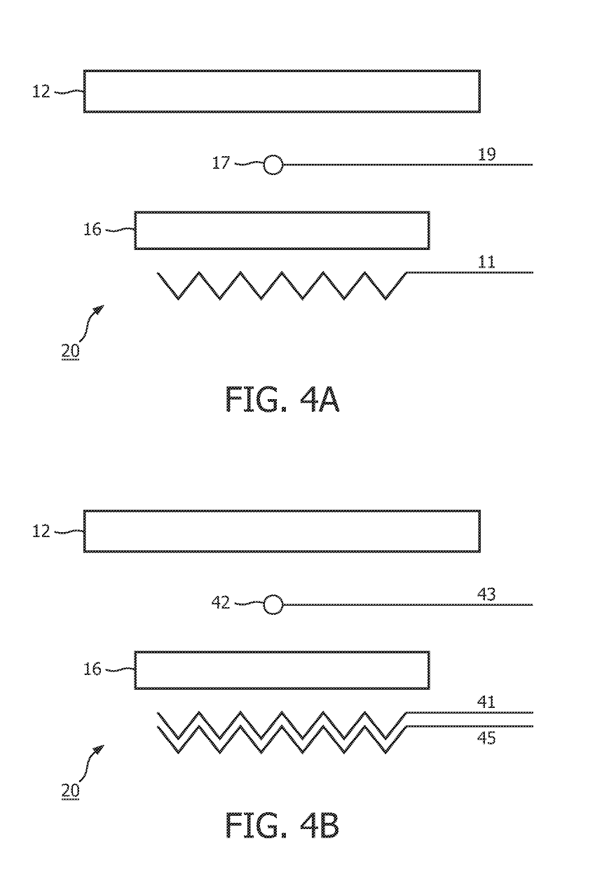

FIGS. 4A-4B schematically illustrate a measuring sub-system in accordance with one or more embodiments;

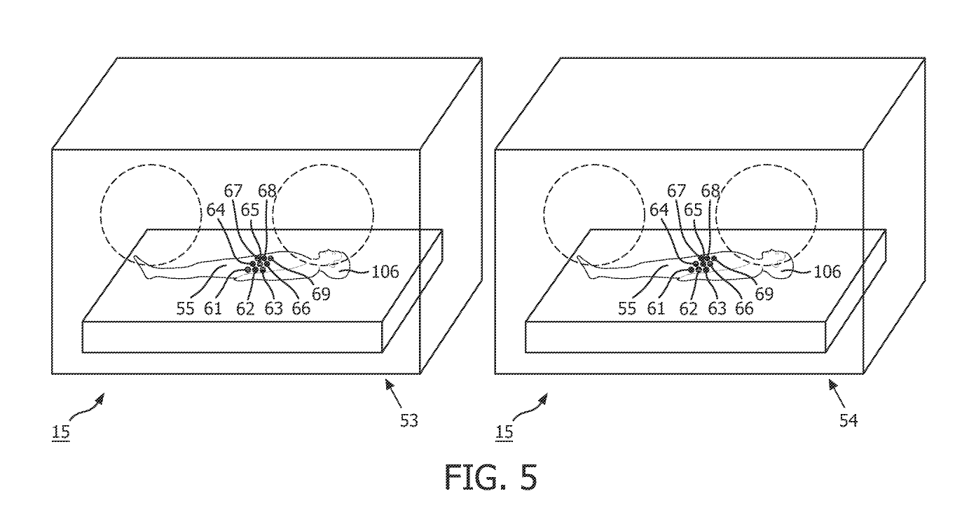

FIG. 5. schematically illustrates a configurable array of temperature sensors integrated in a subject support structure within an incubator;

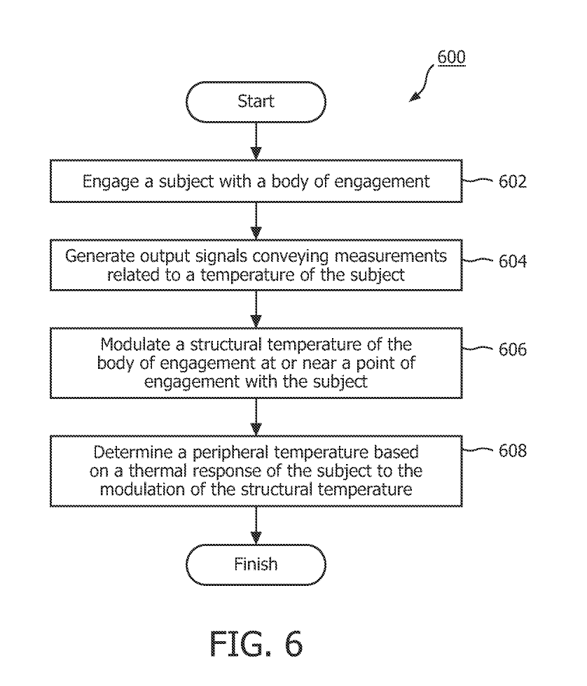

FIG. 6 illustrates a method for measuring a temperature of a subject.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIG. 1 schematically illustrates a sensor 17 integrated in a subject support structure 16 within an incubator 15. Body of engagement 14 is configured to engage with a subject 106, e.g. a neonate and/or infant. In some embodiments, body of engagement 14 may be implemented as a subject support structure 16 configured to support subject 106 thereon. Subject support structure 16 may be a mattress, a pad, a blanket, and/or other structure suitable to support a subject 106, e.g. a neonate and/or infant. In some embodiments, body of engagement 14 may be an article of clothing configured to be worn by subject 106. Sensor 17 may be coupled to a measuring system via a sensor interface 19. Body of engagement 14 (e.g. subject support structure 16) may be configured to carry one or more sensors, e.g. sensor 17. Incubator 15 may include manual access windows 18.

Measuring the core body temperature and/or the peripheral temperature of a subject may be important in many clinical situations, including but not limited to neonates in a neonatal intensive care unit (NICU). Adhesive temperature sensors may damage the skin and cause stress and/or pain when used. Additionally, it may upset a parent or other caretaker to see a baby covered with sensors and/or wires. Integrating sensor 17 within body of engagement 14, subject support structure 16, and/or incubator 15 may provide for contact-less, non-invasive determination of a temperature of subject 106. “Contact-less” refers to either refraining from the use of adhesives and/or refraining from direct skin contact in the context of this disclosure. Other implementations of body of engagement 14 that allow a sensor 17 to measure a temperature of subject 106 are contemplated. References to subject support structure 16 made herein are not intended to be limiting in scope. Rather, subject support structure 16 is referenced as an exemplary embodiment of body of engagement 14. Though sensor 17 is depicted and referred to as one sensor, the disclosure is not limited to one sensor. Sensor 17 may comprise one or more sensors, as well as multiple sensors of different types and capabilities.

Subject 106 may be placed inside incubator 15, e.g. on subject support structure 16, to enable temperature measurements. Sensor 17 may be used to measure the core body temperature of subject 106. Subject support structure 16 may thermally insulate subject 106 from the environment such that temperature measurements taken at or near a point of engagement between subject 106 and subject support structure 16 may (gradually) approximate the core body temperate of subject 106. The thermal principle at work here is known as the zero-heat flux principle, which may be described, e.g., in one or more related applications incorporated by reference into the present application. As a result, and for the same reason, contact-less, non-invasive determination of the peripheral temperature (and/or the difference between the core body temperature and the peripheral temperature) is problematic to obtain: the subject support structure provides thermal insulation to the skin; therefore the skin may not be cooled by the environment and the measured skin temperature may not be representative of what would normally be considered peripheral temperature. “Measure” refers to any combination of measuring, estimating, and/or approximating based on output generated by one or more sensors in the context of this disclosure.

FIG. 2 schematically illustrates a measuring system 10 in accordance with one or more embodiments. Measuring system 10 may be used to measure a temperature of a subject 106 (not shown in FIG. 2). Measuring system 10 may include one or more of a measurement sub-system 20, one or more processors 110, a user interface 120, electronic storage 130, and/or other constituent components. Also illustrated in FIG. 2 is a user 108 of measuring system 10.

Measurement sub-system 20 may include one or more of body of engagement 14 (e.g. subject support structure 16), sensor 17, thermal exchanger 11, and/or other components. Subject support structure 16 is configured to support subject 106 (not shown in FIG. 2) thereon. Sensor 17 is configured to generate output signals conveying measurements related to a temperature of subject 106. Sensor 17 may be carried by subject support structure 16. By locally changing the temperature at or near a point of engagement between subject 106 and, e.g., subject support structure 16, a thermal response by subject 106 may be provoked. This thermal response may be measured through measurement sub-system 20. This measurement may be a basis for determining a peripheral temperature of subject 106 and/or other temperature parameters useful for diagnostic purposes. For example, the peripheral temperature (or a parameter based thereon) may be used to evaluate the status of vasoconstriction and/or perfusion.

Thermal exchanger 11 is configured to exchange thermal energy with body of engagement 14 (e.g. subject support structure 16), e.g. by (locally) modulating a structural temperature, e.g. of subject support structure 16, at or near a point of engagement with subject 106 (not shown in FIG. 2). For example, thermal exchanger 11 may comprise a cooling component. Sensor 17 may be configured to generate one or more output signals conveying measurements related to (gravitational) pressure exerted on sensor 17 by subject 106. Subject support structure 16 may have thermal properties suitable to shield sensor 17 from direct and/or immediate influence by thermal activity of thermal exchanger 11. To properly establish a thermal response of subject 106 to a modulation of the structural temperature by thermal exchanger 11, the thermal resistance of subject support structure 16 may need to be designed as (significantly) larger than the thermal resistance of the materials between the skin (a.k.a. body periphery) of subject 106 and sensor 17.

Returning to measuring system 10 of FIG. 2, measuring system 10 may include electronic storage 130 comprising electronic storage media that electronically stores information. The electronic storage media of electronic storage 130 includes one or both of system storage that is provided integrally (i.e., substantially non-removable) with measuring system 10 and/or removable storage that is removably connectable to measuring system 10 via, for example, a port (e.g., a USB port, a FireWire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 stores software algorithms, information determined by processor 110, information received via user interface 120, and/or other information that enables measuring system 10 to function properly. For example, electronic storage 130 may record or store one or more parameters derived from output signals measured by one or more sensors (as discussed elsewhere herein), and/or other information. Electronic storage 130 may be a separate component within measuring system 10, or electronic storage 130 may be provided integrally with one or more other components of measuring system 10 (e.g., processor 110).

Measuring system 10 may include user interface 120 configured to provide an interface between measuring system 10 and a user (e.g., user 108, a caregiver, a therapy decision-maker, etc.) through which the user can provide information to and receive information from measuring system 10. This enables data, results, and/or instructions and any other communicable items, collectively referred to as “information,” to be communicated between the user and measuring system 10. Examples of interface devices suitable for inclusion in user interface 120 include a keypad, buttons, switches, a keyboard, knobs, levers, a display screen, a touch screen, speakers, a microphone, an indicator light, an audible alarm, and a printer. Information is e.g. provided to subject 106 by user interface 120 in the form of auditory signals, visual signals, tactile signals, and/or other sensory signals.

By way of non-limiting example, in certain embodiments, user interface 120 includes a radiation source capable of emitting light. The radiation source includes one or more of an LED, a light bulb, a display screen, and/or other sources. User interface 120 may control the radiation source to emit light in a manner that conveys to subject 106 information related to, e.g., a breaching of a predetermined temperature threshold by subject 106.

It is to be understood that other communication techniques, either hard-wired or wireless, are also contemplated herein as user interface 120. For example, in one embodiment, user interface 120 is integrated with a removable storage interface provided by electronic storage 130. In this example, information is loaded into measuring system 10 from removable storage (e.g., a smart card, a flash drive, a removable disk, etc.) that enables the user(s) to customize the implementation of measuring system 10. Other exemplary input devices and techniques adapted for use with measuring system 10 as user interface 120 include, but are not limited to, an RS-232 port, RF link, an IR link, modem (telephone, cable, Ethernet, internet or other). In short, any technique for communicating information with measuring system 10 is contemplated as user interface 120.

Processor 110 is configured to provide information processing capabilities in measuring system 10. As such, processor 110 includes one or more of a digital processor, an analog processor, a digital circuit designed to process information, an analog circuit designed to process information, a state machine, and/or other mechanisms for electronically processing information. Although processor 110 is shown in FIG. 2 as a single entity, this is for illustrative purposes only. In some implementations, processor 110 includes a plurality of processing units.

As is shown in FIG. 2, processor 110 is configured to execute one or more computer program modules. The one or more computer program modules include one or more of a control module 111, a parameter determination module 112, an assessment module 113, a measurement module 114, and/or other modules. Processor 110 may be configured to execute modules 111, 112, 113, and/or 114 by software; hardware; firmware; some combination of software, hardware, and/or firmware; and/or other mechanisms for configuring processing capabilities on processor 110.

It should be appreciated that although modules 111, 112, 113, and 114 are illustrated in FIG. 2 as being co-located within a single processing unit, in implementations in which processor 110 includes multiple processing units, one or more of modules 111, 112, 113, and/or 114 may be located remotely from the other modules. The description of the functionality provided by the different modules 111, 112, 113, and/or 114 described below is for illustrative purposes, and is not intended to be limiting, as any of modules 111, 112, 113, and/or 114 may provide more or less functionality than is described. For example, one or more of modules 111, 112, 113, and/or 114 may be eliminated, and some or all of its functionality may be provided by other ones of modules 111, 112, 113, and/or 114. Note that processor 110 may be configured to execute one or more additional modules that may perform some or all of the functionality attributed below to one of modules 111, 112, 113, and/or 114.

Measurement module 114 is configured to control operation of measurement sub-system 20 in the provision of temperature measurements of subject 106. Measurement module 114 may direct operation of other modules of processor 110. Measurement sub-system 20 may operate without activating thermal exchanger 11. In this case, temperature measurements via sensor 17 may gradually approximate the core body temperature of subject 106 due to thermal insulation of sensor 17 by, e.g., subject support structure 16. Alternatively, and/or alternately, measuring sub-system 20 may operate whilst activating thermal exchanger 11 (e.g. through control module 111 as described below). In this case, a modulation of the structural temperature at or near the point of engagement between subject 106 and subject support structure 16 may provoke a (localized) thermal response by subject 106. Sensor 17 may be used to measure the thermal response of subject 106, e.g. the amount of thermal change, the rate of thermal change, and/or both.

By way of illustration, FIG. 3 schematically illustrates a thermal model 30 in accordance with one or more embodiments, during operation of thermal exchanger 11 as a cooling component. The temperature of thermal exchanger is T_{cool}. The body core 32 temperature is T_{core}. The peripheral temperature of skin 12 (of subject 106) is Tₚ, as measured by sensor 17. Subject support structure 16 separates skin 12 from thermal exchanger 11. Thermal model 30 follows the following (static) thermal equation:

$${T}_{p} = \frac{{R}_{i}}{{R}_{i} + {R}_{p}} {T}_{core} + \frac{{R}_{p}}{{R}_{i} + {R}_{p}} {T}_{cool}$$

Rₚ and Rᵢ represent the thermal resistance of the body periphery (i.e. skin) of subject 106 and the (insulating) body of engagement 14 (e.g. subject support structure 16), respectively. A certain range of values for Rₚ may correspond with certain medical conditions, e.g. low perfusion of the body periphery. Additional insulation between skin 12 and sensor 17, e.g. bed sheets and/or clothing, may be represented in thermal model 30 as an additional thermal resistance in series to Rₚ, and accounted for accordingly. A dynamic thermal equation may incorporate thermal capacitances, i.e. for body periphery and/or the subject support structure.

Returning to measurement module 114 and measurement sub-system 20 of FIG. 2, control of the temperature modulation through thermal exchanger 11 may be configured to mimic environmental conditions to provide an intuitive and/or standardized measure of a peripheral temperature. Control of the temperature modulation may be configured to create a predetermined (standardized) thermal environment for measurements, in particular for diagnostic evaluations. Referring to thermal model 30 of FIG. 3, for example, T_{cool} may be controlled such that Tₚ reaches a particular value, a particular difference with T_{core}, and/or another predetermined thermal condition, suitable for evaluating Rₚ or, more precisely, for evaluating the perfusion of the body periphery at or near the location of the measurement.

By way of illustration FIGS. 4A-4B schematically illustrate a measuring sub-system in accordance with one or more embodiments. FIG. 4A illustrates a measurement sub-system 20 including one or more of thermal exchanger 11, subject support structure 16, sensor 17 (separated from thermal exchanger 11 by subject support structure 16), skin 12 (of subject 106), sensor interface 19 (which may enable communication from sensor 17 to measurement module 114 and/or other components of measuring system 10) and/or other components. FIG. 4B illustrates a measuring sub-system 20 including one or more of a cooling component 41, a heating component 45, a subject support structure 16, a thermal flux sensor 42, a sensor interface 43 (which may enable communication from thermal flux sensor 42 to measurement module 114 and/or other components of measuring system 10), skin 12 (of subject 106), and/or other components. The relative placement of cooling component 41 and heating component 45 in FIG. 4B is not intended to be limiting. Alternately cooling and heating the same local area of a subject support structure may provoke a thermal response that can be determined faster than through cooling alone. Measurements from thermal flux sensor 42 may be used to evaluate the efficiency of the thermoregulatory system of subject 106. Different modes of operation may correspond to different levels of heat flux, as measured by thermal flux sensor 42 and communicated to other components of measuring system 10 via sensor interface 43.

In some implementations, measurement sub-system 20 includes a thermal exchanger that comprise one or more of a cooling component, a heating component, and/or a component capable of both cooling and heating (e.g. a Peltier device).

Returning to FIG. 2, control module 111 is configured to control thermal exchanger 11 to modulate a (localized) structural temperature of the subject support structure at or near one or more points of engagement between the subject and the structural support structure. Thermal exchanger 11 may affect more than one local area/region of the subject support structure, and may affect different areas differently. The location of one or more areas of the subject support structure affected by thermal exchanger 11 may correspond to the location of one or more (temperature, pressure, and/or thermal flux) sensors. An array of two or more sensors may be integrated in or carried by a subject support structure.

By way of illustration, FIG. 5. schematically illustrates an exemplary configurable sensor array 55 integrated in a subject support structure 16 within an incubator 15. The sensors in sensor array 55 may be arranged in a grid or any other pattern. Using a sensor array 55 may obviate the need to place the infant on a precise location of subject support structure 16, and/or may account for an infant moving, wriggling, etc. As shown in FIG. 5, sensor array 55 includes nine sensors: 61-69. Operational mode 53 indicates a mode of operation for sensor array 55 in which sensor 61, sensor 63, sensor 65, sensor 67, and sensor 69 are configured and/or used to measure core body temperature, while sensor 62, sensor 64, sensor 66, and sensor 68 are configured and/or used to measure peripheral temperature and/or evaluate tissue perfusion. Contrarily, operational mode 54 indicates a mode of operation for sensor array 55 in which sensor 61, sensor 63, sensor 65, sensor 67, and sensor 69 are configured and/or used to measure peripheral temperature and/or evaluate tissue perfusion, while sensor 62, sensor 64, sensor 66, and sensor 68 are configured and/or used to measure core body temperature. Sensor array 55 may be reconfigured from operational mode 53 to operational mode 54 and vice versa. Reconfiguration may occur automatically, intermittently, manually, and/or according to any programmed schedule. Reconfiguration may occur based on measurements from a pressure sensor and/or based on information other means of assessing the subject location (e.g. through video analysis). The number of sensors and their configuration in sensor array 55 is not limited to the illustrative example of FIG. 5.

Returning to FIG. 2, parameter determination module 112 is configured to determine a thermal response of the subject to a modulation of a structural temperature based on the one or more output signal. Operation of parameter determination module 112 may comprise determining one or more temperature parameters from the output signals generated by sensor 17. The output signals may convey measurements related to pressure exerted on a sensor. The one or more temperature parameters may include peripheral temperature, a temperature difference relative to the core body temperature, a temperature change, a rate of temperature change, the core body temperature, and/or any parameters derived therefrom. Parameter determination module 112 may be configured to determine whether any additional thermal resistance, e.g. bed sheets and/or clothing, is present between the skin of the subject and sensor 17. Alternatively, and/or simultaneously, the presence of additional thermal resistance, e.g. bed sheets and/or clothing, might be indicated by a user and subsequently used by parameter determination module 112 and/or any other component of measuring system 10. Some or all of the stated functionality of parameter determination module 112 may be incorporated or integrated into or controlled by other computer program modules of processor 110.

Assessment module 113 may be configured to assess a diagnosis of conditions related to the temperature and/or temperature management of a subject, including, but not limited to, level of perfusion, vasoconstriction, cardiovascular issues, and/or hypothermia. Assessment may be based on information from parameter determination module 112, output signals from sensor 17, user input and/or other constituent components of measuring system 10.

FIG. 6 illustrates method 600 for measuring a temperature of a subject. The operation of method 600 presented below is intended to be illustrative. In certain embodiments, method 600 may be accomplished with one or more additional operations not described, and/or without one or more of the operations discussed. Additionally, the order in which the operations of method 600 are illustrated in FIG. 6 and described below is not intended to be limiting.

In certain embodiments, method 600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 600.

At an operation 602, a subject is engaged with a body of engagement. In one embodiment, operation 602 is performed using a body of engagement similar to or substantially the same as subject support structure 16 (shown in FIG. 2 and described above).

At an operation 604, output signals conveying measurements related to a temperature of a subject are generated. In one embodiment, operation 604 is performed using a sensor similar to or substantially the same as thermal sensor 17 (shown in FIG. 2 and described above).

At an operation 606, a structural temperature of the body of engagement is modulated at or near the point of engagement with the subject. In one embodiment, operation 606 is performed using a thermal exchanger similar to or substantially the same as thermal exchanger 11 (shown in FIG. 2 and described above).

At an operation 608, a peripheral temperature of the subject is determined based on a thermal response of the subject to the modulation of the structural temperature. In one embodiment, operation 608 is performed using a parameter determination module similar to or substantially the same as parameter determination module 112 (shown in FIG. 2 and described above).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the embodiments have been described in detail for the purpose of illustration based on what is currently considered to be most practical and preferred, it is to be understood that such detail is solely for that purpose and not to pose any limits, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A measuring system for contact-less, non-invasive determination of a temperature of a subject, comprising:
a body of engagement configured to engage with a subject; one or more sensors that generate one or more output signals conveying measurements related to a temperature of the subject, wherein the one or more sensors are carried by the body of engagement;
a thermal exchanger configured to exchange thermal energy with the body of engagement and/or the subject; and
one or more processors configured to execute computer program modules, the computer program modules comprising:
a control module configured to control the thermal exchanger to modulate a structural temperature of the body of engagement at or near a point of engagement between the subject and the body of engagement; and
a parameter determination module configured to determine a peripheral temperature of the subject based on a thermal response of the subject to the modulation of the structural temperature indicated by the one or more output signals.

2. The measuring system of claim 1, wherein the body of engagement is configured to support the subject thereon.

3. The measuring system of claim 1, wherein the body of engagement is configured to be worn by the subject.

4. The measuring system of claim 1, wherein the thermal exchanger comprises a cooling component.

5. The measuring system of claim 1, wherein the thermal exchanger comprises a heating component.

6. The measuring system of claim 1, wherein the control module is configured to control the thermal exchanger to modulate the structural temperature such that the modulation corresponds to a predetermined thermal condition suitable for evaluating perfusion of the subject, based on the thermal response, at or near the point of engagement.

7. The measuring system of claim 1, wherein the control module is configured to control the thermal exchanger to modulate the structural temperature such that the modulation corresponds to a predetermined thermal condition suitable for assessing a thermoregulatory response of the subject, based on the thermal response, at or near the point of engagement.

8. The measuring system of claim 1, comprising a plurality of sensors arranged in a grid, wherein the parameter determination module is configured to determine a peripheral temperature for at least one sensor in the plurality of sensors and a core body temperature for at least one sensor in the plurality of sensors.

9. A method of contact-less, non-invasive determination of a temperature of a subject, the method comprising;
engaging a subject with a body of engagement;
generating one or more output signals conveying measurements related to a temperature of the subject;
modulating a structural temperature of the body of engagement at or near a point of engagement between the subject and the body of engagement; and
determining a peripheral temperature of the subject based on a thermal response of the subject to the modulation of the structural temperature indicated by the one or more output signals.

10. The method of claim 9, the body of engagement is configured to support the subject thereon.

11. The method of claim 9, the body of engagement is configured to be worn by the subject.

12. The method of claim 9, wherein modulating a structural temperature comprises cooling the structural temperature at or near a point of engagement between the subject and the subject support structure.

13. The method of claim 9, wherein modulating a structural temperature comprises heating the structural temperature at or near a point of engagement between the subject and the subject support structure.

14. A system configured for contact-less, non-invasive determination of a temperature of a subject, the system comprising:
means for engaging a subject;
means for generating one or more output signals conveying measurements related to a temperature of the subject;
means for modulating a structural temperature of the means for engaging the subject at or near a point of engagement with the subject; and
means for determining a peripheral temperature based on a thermal response of the subject to the modulation of the structural temperature indicated by the one or more output signals.

15. The system of claim 14, wherein the means for engaging a subject is configured to support the subject thereon.

16. The system of claim 14, wherein the means for engaging a subject is configured to be worn by the subject.

17. The system of claim 14, wherein the means for modulating a structural temperature comprises means for cooling the structural temperature at or near a point of engagement between the subject and the means for engaging the subject.

18. The system of claim 14, wherein the means for modulating a structural temperature comprises means for heating the structural temperature at or near a point of engagement between the subject and the means for engaging the subject.
